(19) United States
(12) Patent Application Publication
(10) Pub. No.: US 2005/0079198 A1
(43) Pub. Date: Apr. 14, 2005

(54) Chitosan-coated metallic article, and process for the production thereof

(76) Inventors: Berthold Nies, Fraenkisch-Crumbach (DE); Andreas Sewing, Darmstadt (DE); Dieter Scharnweber, Dresden (DE); Hartmut Worch, Dresden (DE); Rene Beutner, Dresden (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/918,743

(22) Filed: Aug. 16, 2004

(30) Foreign Application Priority Data
Aug. 15, 2003 (DE) 103 38 110.4

(51) Int. Cl. A61N 1/00; A61F 2/00

(52) U.S. Cl. 424/423; 607/2

(57) ABSTRACT

The invention describes an article made from chitosan-coated metal, where an impermeable chitosan layer having a barrier action is present on the metal through electrochemical polarisation in a chitosan-containing solution. The incorporation of biologically active components into the chitosan coating enables the coating to be matched to various applications and the bio-compatibility of correspondingly modified surfaces to be increased. Metallic articles or surfaces coated in this way are used, for example, as material for medical implants.

The invention relates to a metallic article having a stable coating of chitosan and to a process for the production thereof. The incorporation of biologically active components into the chitosan coating enables the latter to be matched to a very wide variety of applications and the biocompatibility of correspondingly modified surfaces to be increased.

Metallic articles or surfaces coated in this way are of interest for medicine and veterinary medicine, for example for implants, but also for a very wide variety of areas of biotechnology.

The polysaccharide chitosan and modifications of this substance are widely employed in the area of biomaterials for the coating of implants and for the production of scaffolds for tissue engineering.

Thus, U.S. Pat. No. 5,578,073 describes a dip-coating process for the production of chitosan-PVA-based layers on PTFE surfaces for achieving blood coagulation-preventing surfaces through the incorporation of biologically active components into the layer.

WO 96/02259 likewise utilises a dip-coating process for the coating of implant surfaces in contact with bone and adds polysaccharides, such as heparin, heparan sulfate, chondroitin sulfate or dermatan sulfate, to the chitosan solution in order to stimulate the hard tissue reaction.

The production of scaffolds for tissue engineering of bones is achieved by Zhang et a[. (J. of Non-Cryst. Solids 282(2,3), 159-64) through the combination of chitosan with β-tricalcium phosphate.

A similar process is described in EP 0555807, in which a corresponding scaffold is produced by mixing a chitosan sol with pulverulent inorganic bone substance and divalent cations.

CA-2219399, which is directed more to soft-tissue applications, describes the production of monolithic hydrogels based on chitosan and achieves the shaping starting from an acidic solution of the polymer by hydrolysis of an added amide and the resultant neutralisation of the originally acidic solution.

A widely discussed disadvantage in connection with the above-mentioned processes, in particular for the formation of layers, is their inadequate stability.

On this basis, WO 2002080996 combines an extremely resistant, long-term-stable DLC layer, to which the barrier function is ascribed, with a short-term-stable polymer layer (for example made from polylactic acid), into which biologically active ingredients are bound.

In EP 1308177A1, a chitosan layer produced by dip coating is stabilised after drying by storage in stabilisation solution. The stability of the film is increased here via the pH of this solution or the use of covalent crosslinking agents or via combination of the two approaches. In a final process step, the stabilised film is washed.

The loose structure of chitosan films produced by dip coating or solvent casting also follows from investigations by Cruz et al. (Anal. Chem. 72 (2000), 680-86). These authors show, for the transport of, in particular, positively charged metal-ion complexes through chitosan films produced by solvent casting, that the diffusion coefficients are comparable with those for unhindered diffusion in aqueous media.

For numerous applications, simple performance of the process for the production of strongly adherent, impermeable chitosan films having a barrier action on -biomaterial surfaces is therefore of great interest.

The object of the present invention was therefore to provide a metallic article having a stable coating of chitosan in which, in one process step, strongly adherent layers of defined impermeability can be produced, and the way in which the process is carried out provides the possibility of incorporating biologically active components into the layers.

This object is achieved by an article made from metal to the surface of which a chitosan layer is produced from a weakly acidic solution of chitosan with electrochemical support. The addition of biologically active components to the chitosan solution enables incorporation thereof into the layer. Through the electrochemically supported production of the layer, the latter attains an impermeable structure, is strongly adherent and acts as barrier layer between the metallic substrate and the biological environment.

In accordance with the invention, the layers are produced in an aqueous electrolyte by cathodic polarisation of the metallic article to be coated in galvanostatic mode with a current density of between 0.1 and 20 mA/cm², preferably between 2 and 8 mA/cm². The freely selectable electrochemical parameters are the current density, the final potential and the course of the polarisation with time, where the polarisation can be carried out either continuously or pulsed.

The metallic article here can consist of any desired metals or alloys or of any materials employed as metallic biomaterial. The metallic article can have as complex a geometry as desired and can have any desired surface morphology (for example sand-blasted, titanium plasma-coated).

Biologically active components can be incorporated into the chitosan layer produced on the metallic article according to the invention by addition to the coating solution. For the desired case of rapid release of the biologically active component from a loosely structured chitosan film, the process of electrochemically supported layer production can be operated with appropriately selected parameters (preferably low current density and low final potential) and/or extended by a dip-coating process step or storage of the layer in the coating solution without polarisation after the electrochemical polarisation.

Suitable biologically active components are structural or adhesion proteins or a peptide structure derived therefrom. Preference is given to antibiotics, glycosaminoglycans, proteoglycans, cytokines or a structure derived therefrom.

If release of the biologically active component from the chitosan film is not desired, this can advantageously be prevented by bonding of the biologically active component to the chitosan layer. Such bonding can be of an ionic or covalent nature or utilise biological bonds of the biotin/avidin/strepavidin type. Covalent bonding is achieved, for example, by crosslinking using UV light or other chemical reactions.

The invention furthermore relates to a process for the production of the metallic, chitosan-coated article according to Claim 1.

Surprisingly, it has been found that a local increase in the pH as a consequence of cathodic polarisation of a metallic article to be coated in a weakly acidic aqueous solution of chitosan in an organic or inorganic acid results in the formation of a stable, impermeable, strongly adherent chitosan layer. This is evident from the increase in the cell potential during the polarisation. This cell potential achieves, as a function of the selected current density, values >100 V above a limit value in the range from seconds to minutes and is thus an expression of the resultant impermeable layer representing a high ohmic resistance. The electrochemical polarisation is preferably carried out with a cell potential in the range from 20 to 110 V.

In accordance with the invention, the conditions for the layer production are selected in such a way that a 0.1 to 5% solution of chitosan in 0.1 to 5% acid, preferably 1 to 2%, is used. Acids which can be used are both inorganic and organic acids. Preferred organic acids are lactic acid, acetic acid and glutamic acid. Preferred inorganic acids are hydrochloric acid and nitric acid.

The article coated in this way can be sterilised using conventional non-thermal methods, such as ethylene oxide or gamma-irradiation. The choice of sterilisation method here depends essentially on the stability of the biologically active component present in the layer.

If not used immediately subsequently, the sterilised article can be stored at cool temperatures (<10° C.) and with exclusion of light.

A conceivable possible medical application of the coated article according to the invention is bacteriostatic finishing of an implant surface for bone contact by means of a chitosan layer alone or a combination of the chitosan layer with the incorporation of cell adhesion-promoting peptide sequences or proteins, such as, for example, type I collagen.

Another possible application is coating of external fixings, in particular in the region where they pass through the skin, with a bacteriostatically active chitosan layer or a combination of such a layer with inflammation-inhibiting active ingredients incorporated into it.

The process according to the invention is differentiated from known processes by the following main advantages:
a High stability, thickness and structure of the chitosan film produced, which can be adjusted in a defined manner via the choice of the electro-chemical parameters.
Owing to the stability and impermeability of the chitosan film, the latter acts as an effective barrier between the metallic biomaterial and the surrounding tissue in the sense of a reduction in corrosive attack on the metallic biomaterial by constituents of the surrounding tissue or body fluids.
Reduced release of ions from the metallic biomaterial reduces the risk of irritation to the surrounding tissue and/or disadvantageous systemic influences and thus increases the biocompatibility of the construction as a whole.
Reduced release of ions from the the metallic biomaterial as a consequence of the barrier action of the impermeable chitosan coating also allows the use in problem patients of less expensive metals as material for the metallic part of the construction as a whole.
Possibility of specific influencing of the release behaviour and thus the bioavailability of biologically active constituents of the chitosan coating through defined setting of the structure of the chitosan layer via the choice of the electrochemical parameters for the layer production.

The invention furthermore relates to the use of the chitosan-coated article as material for medical implants.

The invention is explained in greater detail with reference to the following working examples. In Examples 1 and 2, layers of average thickness are produced on a titanium alloy or an implant steel. Example 3 documents the production of layers of greater thickness and stability. Example 4 varies the procedure in the layer production to pulsed polarisation, and Example 5 illustrates the incorporation of a biologically active component into the layer structure. Example 6 describes the variant of chitosan solutions in inorganic acids, such as HCl.

### WORKING EXAMPLE 1:

A cylindrical sample of TiAl6V4 having a diameter of 10 mm and a thickness of 3 mm is ground, oxide-polished and washed with ethanol.

A 1% solution of chitosan in 1% lactic acid is prepared by stirring overnight. The cylindrical sample is provided with electrical contacts and dipped into the chitosan solution together with a sheet of stainless steel as counter-electrode.

The electrochemical polarisation is carried out galvanostatically with a current density of 3 mA cm⁻² to a cell voltage of 30 V. With this polarisation, the TiAl6V4 sample is connected as the cathode.

Immediately after completion of the polarisation, the TiAl6V4 sample is removed from the chitosan solution and rinsed with distilled water.

Visually, the sample exhibits a smooth, strongly adherent layer. Both under the light microscope and in scanning electron microscopic investigation, the layer proves to be complete and impermeable. In FTIR spectroscopy, a chitosan spectrum is detected homogeneously on the sample surface.

### WORKING EXAMPLE 2

As in Example 1, but with stainless steel 316L as material to be coated.

### WORKING EXAMPLE 3

As in Example 1, but with a current density of 5 mA cm⁻² to a cell voltage of 100 V.

### WORKING EXAMPLE 4

As in Example 1, but with pulsed polarisation.

### WORKING EXAMPLE 5

A 1% solution of chitosan and 0.1% of tropocollagen in 1% lactic acid is prepared by stirring overnight. Otherwise as in Example 1.

### WORKING EXAMPLE 6

A cylindrical sample of TiAl6V4 having a diameter of 10 mm and a thickness of 3 mm is ground, oxide-polished and washed with ethanol.

A 1% solution of chitosan in 1% HCl is prepared by stirring overnight.

The cylindrical sample is provided with electrical contacts and dipped into the chitosan solution together with a platinum foil counterelectrode.

The cathodic polarisation is carried out galvanostatically with a current density of 5 mA cm⁻² to a cell voltage of 45 V.

Immediately after completion of the polarisation, the TiAl6V4 sample is removed from the chitosan solution and rinsed with distilled water.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosure[s] of all applications, patents and publications, cited herein and of corresponding German application No. 10338110.4, filed Aug. 15, 2003 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Article made from chitosan-coated metal, characterised in that an impermeable chitosan layer having a barrier action is present on the metal through electrochemical polarisation in a chitosan-containing solution

2. Article according to claim 1, characterised in that the electrochemical polarisation is carried out galvanostatically with a current density in the range between 0.1 and 20 mA cm⁻², and the article is polarised as the cathode.

3. Article according to claim 1, characterised in that the electrochemical polarisation is carried out to a cell potential in the range from 5 to 150 V.

4. Article according to claim 1, characterised in that the chitosan-containing solution has a chitosan concentration of from 0.1 to 5%.

5. Article according to claim 1, characterised in that the chitosan-containing solution is an aqueous, weakly acidic solution with an organic or inorganic acid.

6. Article according to claim 5, characterised in that the chitosan-containing solution comprises lactic acid, acetic acid or glutamic acid.

7. Article according to claim 5, characterised in that the chitosan containing solution comprises hydrochloric acid or nitric acid.

8. Article according to claim 1, characterised in that the acid concentration is in the range from 0.1 to 5%.

9. Article according to claim 1, characterised in that the chitosan coating additionally comprises one or more biologically active components.

10. Article according to claim 9, characterised in that at least one of the biologically active components is a structural protein, an adhesion protein or a peptide structure derived therefrom.

11. Article according to claim 9, characterised in that at least one of the biologically active components is an antibiotic.

12. Article according to claim 9, characterised in that at least one of the biologically active components is a glycosaminoglycan, proteoglycan or a structure derived therefrom.

13. Article according to claim 9, characterised in that at least one of the biologically active components is a cytokine.

14. Article according to claim 1, characterised in that the biologically active components are bonded to the chitosan layer after production thereof.

15. Process for the production of an impermeable, metallic, chitosancoated article having a barrier action, characterised in that the metallic article is cathodically polarised in a chitosan-containing solution.

16. Process according to claim 15, characterised in that the electro-chemical polarisation is carried out galvanostatically with a current density in the range between 0.1 and 20 mA cm⁻².

17. Process according to claim 15, characterised in that the electrochemical polarisation is carried out to a cell potential in the range from 5 to 150 V.

18. Process according to claim 15, characterised in that the chitosan-containing solution used is an aqueous, weakly acidic solution with an organic or inorganic acid.

19. Process according to claim 18, characterised in that the organic acid employed is lactic acid, acetic acid or glutamic acid.

20. Process according to claim 18, characterised in that the inorganic acid employed is hydrochloric acid or nitric acid.

21. Process according to claim 15, characterised in that one or more biologically active components are added to the chitosan-containing solution.

22. Process according to claim 21, characterised in that the biologically active components added are a structural protein, adhesion protein or a peptide structure derived therefrom.

23. Process according to claim 21, characterised in that the biologically active component added is an antibiotic.

24. Process according to claim 21, characterised in that the biologically active components added are a glycosaminoglycan, proteoglycan or a structure derived therefrom.

25. Process according to claim 21, characterised in that the biologically active component added is a cytokine.

26. Use of the chitosan-coated article according to claim 1 as material for medical implants.
